(19) United States
(12) Patent Application Publication
(10) Pub. No.: US 2012/0129195 A1
(43) Pub. Date: May 24, 2012

(54) Method of quick laboratory diagnosis if illnesses based on the discovery of specific proteins and equipment for its implementation

(76) Inventors: Artur Martynov, Kharkov (UA); Boris S. Farber, Brooklyn, NY (US); Sonya Sophya Farber, New York, NY (US)

(21) Appl. No.: 12/931,459

(22) Filed: Feb. 1, 2011

Related U.S. Application Data
(63) Continuation of application No. PCT/RU2010/000689, filed on Nov. 22, 2010.

(51) Int. Cl. G01N 33/53; B01D 57/02; G01N 27/327; G01N 21/64; C12Q 1/37

(52) U.S. Cl. 435/7.92; 435/7.1; 435/23; 204/403.14; 204/461

(57) ABSTRACT

A method of quick laboratory diagnosis of diseases, based on the discovery of specific protein targets characteristic to the disease in the reaction of their specific interaction with other protein reagents, distinct in that specific protein targets for the given disease are hydrolyzed with proteolytic enzymes and then the chemical structure of the oligopeptide formula created is modified in such a way that its charge is changed to the opposite. This method of quick laboratory diagnosis of illnesses based on the discovery of specific proteins, and the equipment for its implementation, may be applied for the discovery of viral and microbial antigens in various human biological fluids. The method may also be used for the detection of new and little-studied infectious diseases in connection with the ease of diagnostic preparation.

### TECHNICAL FIELD

This invention is related to medicine—specifically, to methods of laboratory diagnosis—and is designed to replace the existing methods of immune diagnostics with the application of immunoglobulins in the significantly less expensive method being patented.

### PREVIOUS LEVEL OF TECHNOLOGY

Among the existing methods of the laboratory diagnosis of diseases in which specific immunoglobulins are used, the most widespread are the immune-enzyme method and the immunofluorescence method.

The immune-enzyme analysis (the Enzyme-Linked ImmunoSorbent Assay, or ELISA) is a laboratory immunological method for the qualitative determination and quantitative measurement of antigens, as well as of immunoglobulins and hormones. The ELISA's sensitivity and specificity is currently at more than 90%. Test systems for immune-enzyme diagnostics can discover a wide range of different infections: HIV infections, viral hepatitis, cytomegalovirus infections, herpes infections, toxoplasma infections, etc.

The ELISA provides the opportunity to determine the antibodies (IgG, IgA, IgM) to causes of infection in the blood. These antibodies are created by the body in response to infection. The antibodies are discovered when they interact with special substances that contain the corresponding antigens; they create a strong complex with the antibodies that may be discovered according to various methods. The basis of the method is the principle of interaction between an immunosorbent antibody to a cause of a disease with the antibodies discovered. Depending on which antibodies are used, the test system will discover either the specific antibody independent of its class or the antibody of just one given class in the sample being studied. Serum or plasma from venous blood drawn from a fasting subject serve as material for the study.

For the diagnosis of venereal diseases, three classes of immunoglobulins are used: M, A, G.

Due to the fact that immunoglobulins of various classes are created in a certain order, with the help of the ELISA, it is possible to diagnose infections in various stages and follow the dynamic of the process's development. The order of the creation of immunoglobulins of various classes is the following: IgM antibodies appear first. As a rule, this occurs 1-3 weeks from the time of infection. The time for the discovery of the antibodies depends on the infection itself and on the characteristics of a specific person's immune system. Symptoms of severe infection appear. The discovery of IgM antibodies in an analysis indicate the presence of the severe phase of a disease or of a worsening in chronic infectious diseases.

After a month, IgA antibodies begin to form, the main part of which are concentrated in the mucous membranes, where their protective function is carried out. The last to appear are the IgG antibodies, as a rule four weeks after the moment of infection. After treatment of chlamydia, mycoplasms, and trichomoniasis, their level falls significant, as immunity does not develop against these diseases. It should be noted that the discovery of immunoglobulins (IgM, IgG, IgA) indicates only the presence of antibodies, not the presence of the cause of the disease. Therefore, in certain cases, immune-enzyme analysis can also provide both false negatives and false positives. However, the specificity of the best ELISA test systems of the recombinant type is currently approaching 100%.

Therefore, due to the undisputed advantages of immune-enzyme analysis—convenience of the work, speed, objectivity due to the automation of the calculation of the results, the opportunity to study immunoglobulins of various classes (which is important for early diagnosis of illnesses and their prognoses)—at present, it is one of the main methods of laboratory diagnosis. The advantages of the ELISA are the opportunity for early diagnosis of infections, the chance to follow the dynamic of the development process, and the convenience of the work. The disadvantage of the ELISA is related to its indirect method of diagnosis: it allows the determination of the body's immune response to the cause of the disease, but not the determination of the disease itself.

Immunofluorescent analysis is also used for the discovery of both antigens and antibodies. This method is based on the use of reagents that are marked with a fluorescent dye. Antibodies are most often marked with fluorescein isorhodanate. The marked antibodies join with the antigen, forming complexes that may be seen with the help of a fluorescent microscope. There are three modifications to immunofluorescent analysis.

1. The direct immunofluorescence method is used to discover antigens. It is based on the direct fixation of an antigen, occluded on a hard surface, with marked antibodies. The reaction is evaluated using a fluorescent microscope.

2. The indirect immunofluorescence method allows the discovery of an antibody to a known antigen. The antigen, occluded on a hard surface, connects with unmarked antibodies. The compound antigen-antibody is discovered through the use of marked antibodies to immunoglobulins.

3. The method of concurrent immunofluorescence is based on the combination of the standard marked antigens and the unmarked antigens in the test sample to the antibodies fixed on a hard surface. Since the marked and unmarked antigens are competing for bonds with the antibodies, the concentration of the antigen in the test sample can be determined from the number of bonded marked antigens.

A method for the detection of the influenza virus and the components for its use are known [¹] in which the antibody is not used. In this method, components that are capable of specifically bonding with the active sections of the influenza virus's neuramidinase are being patented, as are new components for use in this method. In the capacity of substances that specifically interact with influenza neuraminidase, synthetic derivative analogues to neuramino acids are being patented. The use of the synthetic derivatives of neuraminidases in antibody test systems in place of immunoglobulins is proposed. The shortcomings of the method are the fully synthetic nature of the neuraminidase derivatives and correspondingly the difficulty of synthesizing and producing the test system, which significantly raises its price, the need to synthesize a derivative for every strain of flu virus with its own neuraminidase, and the impossibility of using the method for the detection of other infections and the discovery of proteins other than influenza proteins.

An immune diagnostic method and its equipment for the detection of viral antigens such as herpes simplex are also known [²]. The viral antigen is immunoconjugated with a specific IgG immunoglobulin against a viral antigen and occluded on an insoluble matrix. The matrix interacts upon contact with monoclonal IgM immunoglobulins marked with biotin. The presence of the antigen to the herpes virus is discovered through the reaction of the avidin with the biotin. The method is an analogue to immune-enzyme analysis. A shortcoming of the method is the need to obtain special immunoglobulins for viral antigens through the use of animals or the use of a hybrid for obtaining monoclonal antibodies, which significantly raises the price of the end product. Also, the method uses multiple covalent conjugation of immunoglobulins with biotin and specific immunoglobulins with an insoluble matrix, which significantly reduces the specificity and sensitivity of the immunoglobulins, causing the precision of the tests conducted to be reduced. In addition, this method is designed only for the detection of viral antigens and cannot be used to discover bacterial, fungal, or other low-immunogenic proteins to which it is difficult to induce synthesis of specific immunoglobulins in immunized animals.

A biosensor is known that can detect the presence in biological material of a specifically determined protein; it comprises a film placed on a conductive layer to create an electrode, a protective dielectrical covering, contact leads, an immuno-chemical biomatrix on the surface of one of the electrodes containing sorbed immunoglobulins that are specific to a certain protein [³].

### DISCLOSURE OF THE INVENTION

The basis of this invention is a task that has been set to develop a method of quick laboratory diagnosis of diseases and a device to carry it out, based on the discovery of specific proteins able to discover any protein specific to a certain disease, including low-immunogenic proteins, to which it is difficult or impossible to obtain specific immunoglobulins. In the proposed method, immunoglobulins and animals for producing them are not used, and all the procedures for obtaining the protein reagent comprise a few simple procedures and allow the development of a test system for the discovery of new and little-studied infectious diseases in the shortest term possible.

The task set is addressed through development of a method of quick laboratory diagnosis of diseases, based on the discovery of specific protein targets characteristic to the disease in the reaction of their specific interaction with other protein reagents, distinct in that specific protein targets for the given disease (taken from a microorganism, virus, or microscopic fungus) are hydrolyzed with proteolytic enzymes. The chemical structure of the macromolecular assembly made of oligopeptides that is created is modified through acylation with succinic anhydride or alkylation with monochloracetic acid. The reaction is conducted in such a manner so that their charges change to the opposite, and they are used further in the capacity of protein reagents in immunofluorescent and immune-enzyme methods of analysis or in the use of an impedance biosensor instead of specific and anti-species immunoglobulins. In the capacity of proteolytic enzymes, pepsin, trypsin, papain, and chymotrypsin may be used. Between the macromolecular assembly of acidic oligopeptides obtained and the fluorescing substance of fluorescein isorhodanate or another fluorescent pathfinder, fluorescing conjugates may be obtained for further use in the fluorescing antibody method (direct or indirect immunofluorescence). Analogously, conjugates between the supramolecular assembly of acidic peptides and the peroxidase enzyme may be obtained for further use of the conjugate in immune-enzyme analysis.

The task set is also addressed through the development of a device (equipment) for the quick diagnosis of diseases through the determination of the presence in biological material of a specifically determined protein, which comprises a film placed on its conductive layer to create an electrode, a protective dielectric covering, contact leads, an immuno-chemical biomatrix on the surface of one of the electrodes containing sorbed immunoglobulins specific to the determined protein, distinct in that in the capacity of a biomatrix, the detected antigen is used, affixed to a metallic surface, hydrolyzed with proteolytic enzymes and modified with a change in the molecules' charge to their opposite.

Instead of immunoglobulins, the authors used an assembly of oligopeptides that were the product of the hydrolysis of specific antigens detected as a result, but the molecules' charges were changed to the opposite. “Assembly” is a term from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell [4,5]

The specific interaction is conditioned on the conjugation and hybridization effect of homogeneous peptides, but they have the opposite charge and they aggregate by themselves in the target of a supramolecular assembly. The method permits the exclusion of the stage of obtaining specific immunoglobulins, therefore eliminating the use of animals. It also speeds up the development of test systems for new and little-studied infectious diseases to a period of several days and significantly reduces the cost of test system production while maintaining the sensitivity of the method and substantially increasing its specificity.

### SHORT DESCRIPTION OF DRAWINGS

FIG. 1. A photograph of epithelial smear cells not infected by the herpes virus according to the method being patented

FIG. 2. Cells of the buccal epithelium infected by the herpes type 1 virus, discovered by the method being patented

FIG. 3. The structure (scheme) of the proposed equipment: an impedance biosensor (view from above): 1—electrodes (gold, platinum, iridic, etc.); 2—silicone base; 3—immuno-chemical compartment containing the sorbed peptidohydrolyzate; 4—isolating base (glass, silicone, plastic, rubber, etc.); electrodes are attached to the immuno-chemical compartment on two sides.

FIG. 4. The structure (scheme) of the proposed equipment: an impedance biosensor (side view): 1—electrodes (gold, platinum, iridic, etc.); 2—silicone base; 3—immuno-chemical compartment containing the sorbed peptidohydrolyzate; 4—isolating base (glass, silicone, plastic, rubber, etc.); electrodes are attached to the immuno-chemical compartment on two sides.

FIG. 5. The principle of operation of the proposed device—an impedance multi-electrode immuno-chemical biosensor on a gold base—is as follows: a) there is no reaction between the antibodies and the antigens in the solution; b) precipitation (the creation of a compound and a precipitate) occurs between the antibodies and the antigens in the solution, which is reflected in the nature of the impedance curve.

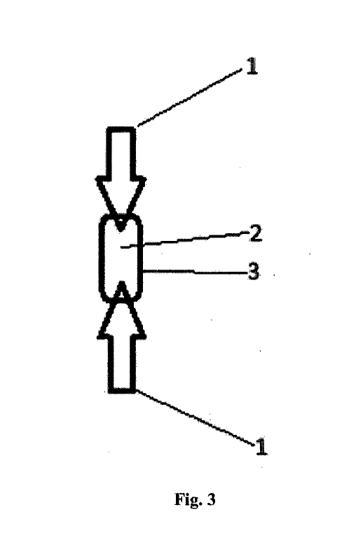

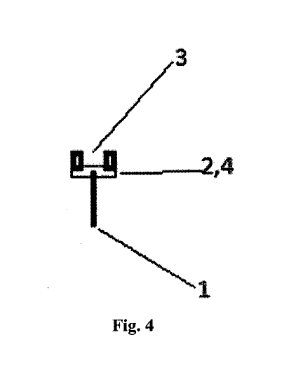

The device works in the following manner (FIG. 3-5). When biological material is introduced to the immuno-chemical compartment (3), over a period of time, the impedance curve changes if there is an immuno-chemical reaction (between the acylated peptides from the surface of the compartment and the specific antigen). In the absence of an immuno-chemical reaction, the impedance curve remains unchanged. This allows the detection of the presence of a specifically determined protein in the biological material.

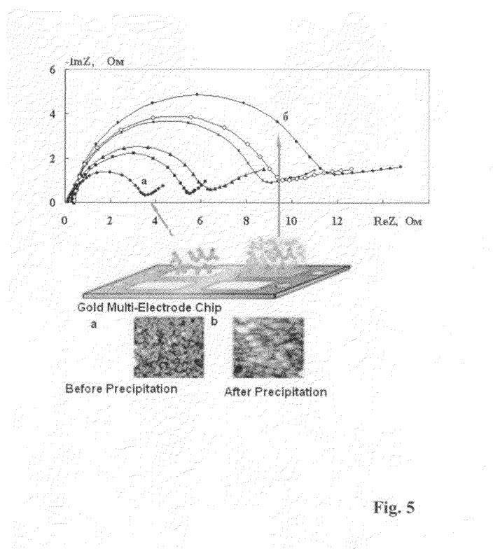

The fastest and most precise methods for the selective detection of DNA, RNA, and proteins are biosensors, whose principle of operation is based on the impedance, admittance, and electronic conductivity of DNA [1-3]. The hardware of detectors has been studied for a long time and has been well developed: the registration of signals takes place through electrochemical methods: chronoamperometry, cyclical voltammetry, and electrochemical impedance spectroscopy (EIS) [4-6]. The main problem in biosensors is the electrode: it either has a limited service time or is so highly sensitive that it provides false negative readings. At present, immunobiosensors are being used with increasing frequency. Their principle of operation is based on the discovery through electrochemistry of the reaction that creates antigen-antibody compounds. The antibody is applied to one of the electrodes (affixed with maleimide or imide silicic acids), and impedance in a control solution is measured. Then the experimental solution containing a determined antigen is applied. If immune compounds form between the antibodies applied to the electrode and the antigens, the voltammetric curve picture changes. The correlation between the quantity of antigens in the sample and the nature of the curve is then easily determined. In an analogous manner, specific antibodies in a solution may also be discovered if a specific antigen is sorbed on the electrode. The shortcoming of this method is the need to use immunoglobulins (antibodies) for the discovery of the antigens. Immunoglobulins are the most expensive component of a biosensor in connection with the fact that in order to obtain an effective immunobiosensor, polyclonal immunoglobulins from a vaccinated animal (most often rabbits) are needed. Accordingly, to obtain industrial quantities of immunoglobulins, a vivarium and a system for the cleansing of blood proteins and the chromatographic separation of immunoglobulins are necessary. The method we are proposing permits the replacement of immunoglobulins with a certain antigen, but with its molecules changed to the opposite charge. Before they are placed on the electrode, these antigens are fragmented using proteolytic enzymes and modified with anhydrides of dicarboxylic acids or with chloride acids. In this case, the sorbed fragments of the antigen acquire complementary and affinity properties to their non-modified derivatives. In addition, unlike hydrogen bonds between immunoglobulins and antigens, ionic bonds form between acylated fragments and native antigen. The reaction takes place very quickly: literally within a few seconds. This permits the creation of biosensors for the instantaneous discovery of microbial and viral agents in biological media in cases of life or death for a patient. FIG. 5 shows impedance for a multi-electrode biosensor chip.

### EXAMPLES OF INVENTION IMPLEMENTATION

### Example 1

### Obtainment of a Test System for the Diagnosis of Influenza Containing Neuraminidase N1 and Hemagglutinin H1

Amniotic fluid is taken after the infection of a chicken embryo with the H1N1 influenza virus and its incubation until the aggregation of the maximum quantity of viruses in known standard conditions. The influenza virus contained therein is then purified according to known method and is concentrated through dialysis. To the concentrate obtained, a solution of trypsin is added so that the ratio of enzyme:protein is 1:100. This is left in an incubator at a temperature of 37° C. for 12 hours. The concentration of dissolved oligopeptides that are the products of hydrolysis was determined via spectrophotometer at 280 nm and 260 nm. The spectrophotometry method of protein determination is based on the ability of aromatic amino acids (tryptophan, tyrosine, and to a lesser extent, phenylalanine) to absorb ultraviolet light, with the maximum absorption at 280 nm. It is conditionally acceptable to believe that at a protein concentration in the solution equal to 1 mg/ml, the optical density value at 280 nm is equal to 1 when cuvettes with a layer thickness of 10 mm are used. The drug's eluent was used in the capacity of a comparison solution. The concentration of the experimental protein in the solution must be from 0.05 to 2 mg/l. The presence of nucleic acids and nucleotides (more than 20%) inhibit the identification of the protein. In this case, the optical density of the same solution is measured at two wavelengths: 260 and 280 nm; the amount of protein X (mg/ml) is calculated using the Calcar formula:

X=1.45·D₂₈₀−0.74·D₂₆₀.

The mixture of oligopeptides and RNA obtained is boiled for 10 minutes; then the sediment of non-hydrolyzed biopolymers that has been created is separated by centrifuge at 5 g over 20 minutes. To the sedimentary liquid, fluorescein isorhonate is added at a ratio to the protein of 1/10000; the solution is left to stand at +5° C. for five hours. Then solid succinic anhydride is added at a ratio to the protein concentration of 2:1. The reagent mixture obtained is used in the antibody fluorescing method. Standard bovine serum albumin conjugated with rhodamine is also used in the array.

### Example 2

### Detection of Infection by the H1N1 Influenza Virus

Bronchial secretions, nasal discharge smears, and blood are taken from patients in whom influenza is suspected. Each sample is resuspended in a 0.9% buffered solution of sodium chloride and centrifuged. The resuspension and centrifuging procedure is repeated three times to clean the cells of accompanying soluble components. The cell sediment is taken up with a micropipette and placed on a slide; with another piece of glass, the cell suspension is spread evenly across the slide. The smear is allowed to dry and fixed with an acetone solution or with a Nikiforov mixture until the smear is desiccated. The peptide formula obtained in Example 1 is then placed on the dried smear and left to incubate at a temperature of 37° C. for 40 minutes in a humid chamber to keep the smear from desiccating. Then the smear is rinsed with a buffered 0.9% solution of sodium chloride, and a 0.1% solution of rhodamine-tagged bovine serum albumin is added; this is left to stand for 20 minutes in the incubator in a humid chamber. The tagged albumin processing is necessary in order to block extra cell epitopes not connected with the specific fluorescing peptides. Then the smear is removed from the incubator, rinsed with distilled water, and dried. Cells fluorescing green are detected under a fluorescent microscope. The cells infected by a virus fluoresce green; the healthy cells fluoresce red. If instead of a glass slide, a Gorjaev's chamber or fluorometric attachment is used, the percentage of cells infected with viruses can be counted.

To test the workability of the method, the test system developed was verified in a comparative test with the standardized, registered IIFM test system, the PCR test system, and the culture method. For detection purposes, tissue samples were obtained from hospital study patients aged 12 to 75 years of both sexes during a flu epidemic.

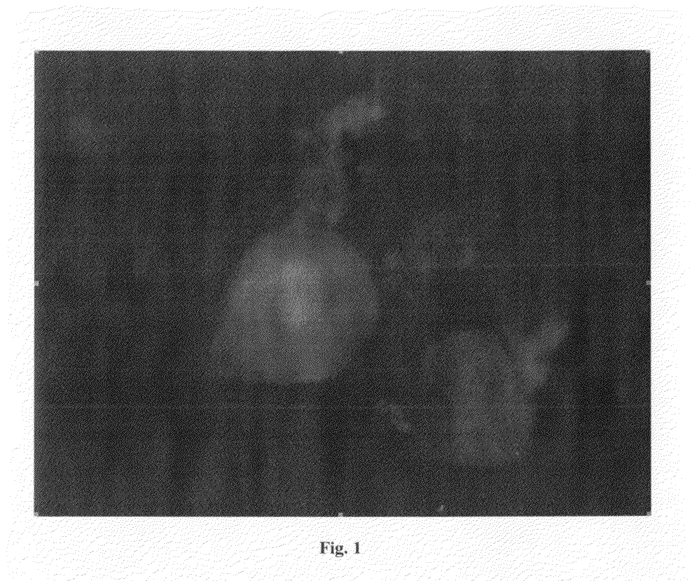

As a control, the standard test system for the indirect immunofluorescent reaction (IIFM) for discovery of the H1N1 virus, made by the National Institute of Influenza Research of the Russian Academy of Medical Sciences (St. Petersburg, Russian Federation), the TaqMan (USA) revertase PCR diagnostic process, and discovery of the virus in ovo with its detection through the standardized hemagglutination method were used. In FIG. 1, the results of the comparison between the standard IIFM and the patented method are presented.

TABLE 1
Comparative Results of the Study of Patients from Two Groups: With Clinical
Symptoms of Influenza and a Control Group without Clinical Symptoms of Influenza
Undergoing Planned Study

| | Viral Antigen Discovered | | Antigen Discovered in | | Antigen Discovered in | |
|---|---|---|---|---|---|---|
| | in Bronchial Secretions | | Smear from Nasal | | Blood Leukocytes | |
| | (Total Patients/ | | Discharge (Total Patients/ | | (Total Patients/ | |
| | Discovered/%) | | Discovered/%) | | Discovered/%) | |
| | | Control | | Control | | Control |
| | Experimental | (without | Experimental | (without | Experimental | (without |
| | Group (with | clinical | Group (with | clinical | Group (with | clinical |
| | clinical symptoms | symptoms | clinical symptoms | symptoms | clinical symptoms | symptoms |
| Method | of flu) | of flu) | of flu) | of flu) | of flu) | of flu) |
| Substance Being | (180/107/59) | (40/4/10) | (180/102/57) | (40/2/5) | (180/110/61) | (40/4/10) |
| Patented | | | | | | |
| Control IIFM | (180/62/34) | (40/1/2.5) | (180/69/38) | (40/0/0) | (180/68/38) | (40/2/5) |
| Control | (180/102/57) | (40/6/15) | (180/100/55) | (40/5/12) | (180/100/55) | (40/4/10) |
| PCR | | | | | | |
| Cultured in ovo, | (180/106/59) | (40/3/7) | (180/101/56) | (40/1/2) | (180/110/61) | (40/4/10) |
| Detection of | | | | | | |
| Hemagglutination | | | | | | |

As may be seen in Table 1, the results of the analysis obtained from the method developed correlate most closely to the gold standard of virology: the culture method of viral detection and the PCR method in both the group of patients with clinical symptoms of influenza and in practically healthy people. Thus the proposed method has a high level of sensitivity and specificity; in accuracy it approaches the culture method of viral detection, which is a standard of viral diagnostics.

### Example 3

### Obtainment of a Test System for the Diagnosis of Human Herpes Simplex Virus Type 1 (HSV-1)

Amniotic fluid is taken after the infection of a chicken embryo with HSV-1 (L-2 strain) and its incubation until the aggregation of the maximum quantity of viruses in known standard conditions. The HSV-1 contained therein is then purified according to known method and is concentrated through dialysis. To the concentrate obtained, a solution of trypsin is added so that the ratio of enzyme:protein is 1:100. This is left in an incubator at a temperature of 37° C. for 12 hours. The concentration of dissolved oligopeptides that are the products of hydrolysis was determined via spectrophotometer at 280 nm and 260 nm, as described in Example 1.

The mixture of oligopeptides and DNA obtained is boiled for 10 minutes; then the sediment of non-hydrolyzed biopolymers that has been created is separated by centrifuge at 5 g over 20 minutes. To the sedimentary liquid, fluorescein isorhonate is added at a ratio to the protein of 1/10000; the solution is left to stand at +5° C. for five hours. Then solid succinic anhydride is added at a ratio to the protein concentration of 2:1. The reagent mixture obtained is used in the antibody fluorescing method. Standard bovine serum albumin conjugated with rhodamine is also used in the array.

### Example 4

### Detection of Infection by HSV-1

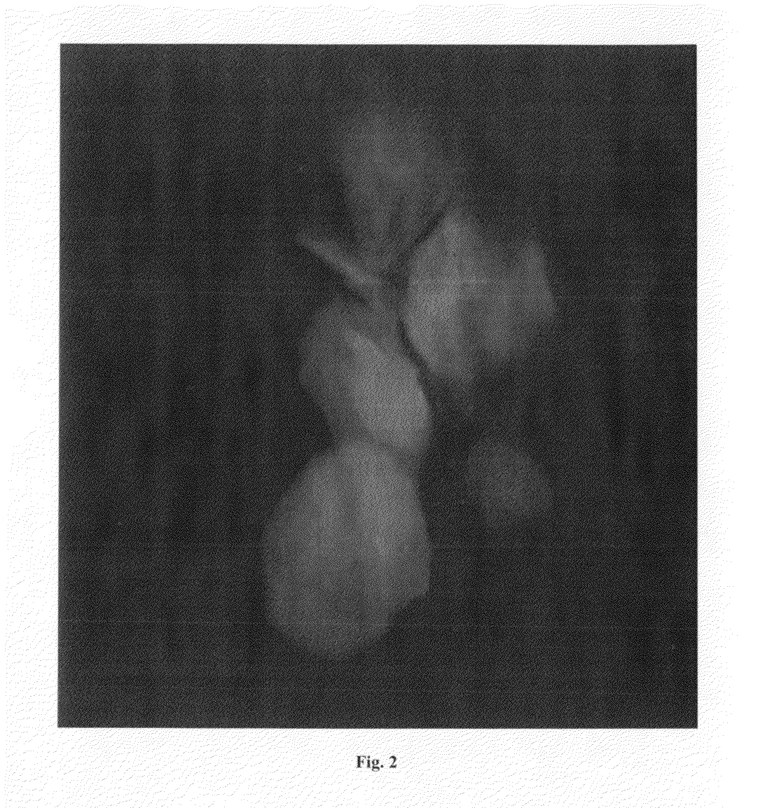

Smears of the lesion focus and the blood leukocytes are taken from patients with signs of labial HSV-1 infection. Each sample is resuspended in a 0.9% buffered solution of sodium chloride and centrifuged. The resuspension and centrifuging procedure is repeated three times to clean the cells of accompanying soluble components. The leucocytes are also detected through the standard method on a gradient of Fikkol/verografin. The cell sediment is taken up with a micropipette and placed on a slide; with another piece of glass, the cell suspension is spread evenly across the slide. The smear is allowed to dry and fixed with an acetone solution or with a Nikiforov mixture until the smear is desiccated. The peptide formula obtained in Example 3 is then placed on the dried smear and left to incubate at a temperature of 37° C. for 40 minutes in a humid chamber to keep the smear from desiccating. Then the smear is rinsed with a buffered 0.9% solution of sodium chloride, and a 0.1% solution of rhodamine-tagged bovine serum albumin is added; this is left to stand for 20 minutes in the incubator in a humid chamber. The tagged albumin processing is necessary in order to block extra cell epitopes not connected with the specific fluorescing peptides. Then the smear is removed from the incubator, rinsed with distilled water, and dried. Cells fluorescing green are detected under a fluorescent microscope. The cells infected by a virus fluoresce green (FIG. 2); the healthy cells fluoresce red (FIG. 1). If instead of a glass slide, a Gorjaev's chamber or fluorometric attachment is used, the percentage of cells infected with viruses can be counted.

To test the workability of the method, the test system developed was verified in a comparative test with the standardized, registered IIFM test system, the PCR test system, and the culture method. For detection purposes, samples were obtained from ambulatory study patients with complaints of labial herpes aged 22 to 60 years of both sexes.

As a control, the standard test system for the indirect immunofluorescent reaction (IIFM) for discovery of HSV-1, made by Labdiagnostika (Moscow, Russian Federation), the NPFDNA-Technology (Russian Federation) PCR diagnostic process, and discovery of the virus in ovo with its detection through the standardized immunofluorescence method by Labdiagnostika were used. In FIG. 1, the results of the comparison between the standard IIFM and the patented method are presented.

TABLE 2
Comparative Results of the Study of Patients from Two Groups: An Experimental
Group with Clinical Symptoms of Labial HSV-1 Infection and a Control Group
without Clinical Symptoms of Labial HSV-1

| | Virus Antigen Discovered in | | | |
|---|---|---|---|---|
| | Smear from Lesion Focus | | | |
| | (Total Patients/Discovered/%) | | | |
| | Experimental | | Antigen Discovered in Leukocytes | |
| | Group (with | Control (with- | (Total Patients/Discovered/%) | |
| | clinical symp- | out clinical | Experimental Group | Control (without clinical |
| | toms of labial | symptoms of | (with clinical symptoms | symptoms of labial |
| Method | herpes) | labial herpes) | of labial herpes) | herpes |
| Substance Being | (43/43/100) | (18/4/10) | (43/43/100) | (18/6/33) |
| Patented | | | | |
| Control IIFM | (43/34/34) | (18/1/2.5) | (43/40/93) | (18/6/33) |
| Control | (43/102/57) | (18/6/15) | (43/36/84) | (18/0/0) |
| PCR | | | | |
| Cultured in ovo, | (43/43/100) | (18/3/7) | (43/12/28) | (18/4/22) |
| Detection IIFM | | | | |

As may be seen in Table 2, the results of the analysis obtained from the method developed correlate most closely to the IIFM and the culture method of viral discovery in both the group of patients with clinical symptoms of labial herpes and in healthy people. Thus the proposed method has a high level of sensitivity and specificity; in accuracy it approaches the IIFM.

### INDUSTRIAL APPLICABILITY OF THE METHOD AND EQUIPMENT

This method of quick laboratory diagnosis of illnesses based on the discovery of specific proteins and equipment for its implementation may be implemented at pharmaceutical and biotechnological companies without changes to the technological cycle, and it does not require new, unique equipment or reactants. The method permits the exclusion of the stage of obtaining specific immunoglobulins, therefore eliminating the use of animals. It also speeds up the development of test systems for new and little-studied infectious diseases to a period of several days and significantly reduces the cost of test system production while maintaining the sensitivity of the method and substantially increasing its specificity. The method correlates in specificity and sensitivity to the culture method of discovery of viruses and polymerase chain reactions, and it does not require complex or expensive equipment.

### REFERENCES

1 U.S. Pat. No. 6,242,582 Method of detection of influenza virus and compounds for use therein//Phillip Reece, Wen-Yang Wu, Betty Jin, Guy Kripper, Keith Watson
2 U.S. Pat. No. 4,535,057 Immunoassay employing monoclonal herpes simplex antibody and biotin-avidin detection system//Gordon Dressman, Cynthia Kendall
³ U.S. Pat. No. 5,567,301
⁴ http://dic.academic.ru/dic.nsf/ruwiki/79240
⁵ Jean-Marie Lehn. Supramolecular Chemistry. Concepts and Perspectives.—Weinheim; N.Y.; Basel; Cambridge; Tokyo: VCH Verlagsgesellschaft mbH, 1995.-P. 103 (Chapter 7)

1. A method of quick laboratory diagnosis of diseases, based on the discovery of specific protein targets characteristic to the disease in the reaction of their specific interaction with other protein reagents, distinct in that specific protein targets for the given disease are hydrolyzed with proteolytic enzymes and then the chemical structure of the oligopeptide mixture (assembly) created is modified in such a way that its charge is changed to the opposite; they are further used in the capacity of protein reagents.

2. A method according to claim 1, distinct in that before changing the molecular charge in the oligopeptide compound, they are covalently modified by a fluorescing agent; the chemical structure of the oligopeptide mixture (assembly) created is then covalently modified with a change in its charge to the opposite and used in a method of immunofluorescent analysis.

3. A method according to claim 1, distinct in that before changing the molecular charge in the oligopeptide compound, they are covalently modified by a bivalent linking agent; the chemical structure of the oligopeptide mixture (assembly) created is then covalently modified with a change in its charge to the opposite; further, a marker enzyme is added, which creates a conjugate of the enzyme, and the peptide compound is used in a method of immune-enzyme analysis.

4. A method according to claim 1, distinct in that in the capacity of a proteolytic enzyme, trypsin is used.

5. A method according to claim 1, distinct in that in the capacity of a proteolytic enzyme, papain is used.

6. A method according to claim 1, distinct in that in the capacity of a proteolytic enzyme, chymotrypsin is used.

7. A method according to claim 1, distinct in that in the capacity of a proteolytic enzyme, pepsin is used.

8. A method according to any one of claims 4-7, distinct in that the product of the reaction between the compound of modified peptides and the specific protein target is discovered through gel electrophoresis by the increase in the mass of the protein target.

9. A method according to claim 2, distinct in that in the capacity of a fluorescent agent, fluorescent low rhodanic ester is used.

10. A method according to claim 2, distinct in that in the capacity of a fluorescent agent, rhodamine is used.

11. A method according to claim 3, distinct in that in the capacity of a marker enzyme, peroxidase is used.

12. A method according to claim 3, distinct in that in the capacity of a bivalent linking agent, glutaric dialdehyde is used.

13. A method according to claim 3, distinct in that in the capacity of a bivalent linking agent, maleimide is used.

14. A method according to claim 3, distinct in that in the capacity of a bivalent linking agent, malondialdehyde is used.

15. A method according to claim 1, distinct in that the covalent modification of the structure of the formula of oligopeptides with a change in charge is performed using succinic anhydride.

16. A method according to claim 1, distinct in that the covalent modification of the structure of the formula of oligopeptides with a change in charge is performed using monochloracetic acid.

17. A method according to claim 1, distinct in that in the capacity of a protein target, a microorganism inactivated by temperature or radiation is used.

18. A method according to claim 1, distinct in that in the capacity of a protein target, an individual protein from a microorganism inactivated by temperature or radiation is used.

19. A method according to any one of claims 17-18, distinct in that in the capacity of a microorganism, viruses are used.

20. A method according to any one of claims 17-18, distinct in that in the capacity of a microorganism, bacteria are used.

21. A method according to any one of claims 17-18, distinct in that in the capacity of a microorganism, microscopic fungi are used.

22. A device (equipment) for the implementation of a method for the quick diagnosis of diseases with detection through the determination of the presence in biological material of a specifically determined protein, which comprises a film placed on its conductive layer, creating an electrode, a protective dielectric covering, contact leads, an immuno-chemical biomatrix on the surface of one of the electrodes containing sorbed immunoglobulins specific to the determined protein, distinct in that in the capacity of a biomatrix, the detected antigen is used, affixed to a metallic surface, hydrolyzed with proteolytic enzymes and modified with a change in the molecules' charge to their opposite.
